(19) United States
(12) Patent Application Publication
(10) Pub. No.: US 2005/0271657 A1
(43) Pub. Date: Dec. 8, 2005

(54) Intermittent treatment regime for organ failure

(75) Inventors: Dianna L. Devore, Windsor (AU); David Norman Haylock, Melbourne (AU)

Correspondence Address: BOZICEVIC, FIELD & FRANCIS LLP, 1900 UNIVERSITY AVENUE, SUITE 200, EAST PALO ALTO, CA, 94303, US

(73) Assignee: Australian Stem Cell Centre Ltd.

(21) Appl. No.: 10/928,633

(22) Filed: Aug. 26, 2004

Related U.S. Application Data
(60) Provisional application No. 60/578,153, filed on Jun. 8, 2004.

(51) Int. Cl. G01N 33/567; A61K 31/401; A61K 39/395

(52) U.S. Cl. 424/144.1; 514/423; 435/7.21

(57) ABSTRACT

A method of treating patients with coronary heart failure (CHF) by the intermittent administration of Angiotensin Converting Enzyme (ACE) inhibitors and/or angiostatin II receptor agonists (ARBs) is disclosed. The ACE inhibitor is administered to the patient during a treatment period and the levels of hematopoietic stem cells (HSC) in the patient's blood is determined. Treatment with the ACE inhibitor is discontinued (during a recovery period) when the measured level of hematopoietic cells drops below a given point and is re-administered when levels rise above a given point. Alternating, treatment and recovery periods can be repeated a plurality of times. Levels of HSC may be determined based on ACE⁺ cell levels or by monitoring CD34 levels and the results used to determine optimum points for the administration of drugs which reduce HSC levels.

### CROSS-REFERENCE

This application claims the benefit of U.S. Provisional Application No. 60/578,153, filed Jun. 8, 2004, which application is incorporated herein by reference.

### FIELD OF THE INVENTION

The invention relates to the field of treating patients with organ failure (e.g., congestive heart failure (CHF) and end-stage renal disease) via a treatment regime utilizing modification of the renin-angiotensin system and regeneration via introduction of hematopoietic progenitor cells.

### BACKGROUND OF THE INVENTION

Specific renin-angiotensin systems have been observed to be present within the cells of specific organ systems such as, for example, the kidney, heart, brain, and blood vessels. This pathway has become a very important target for treating disease including hypertension, heart failure, and renal failure. Modulation of the renin-angiotensin system is central to the treatment regime for congestive heart failure (CHF) and end stage renal failure, as decrease in hypertension relieves the burden on the ailing organ.

In the renin-angiotensin system, the angiotensin converting enzyme (ACE) converts the octapeptide angitensin I to angiotensin II (AII) by cleavage of a dipeptide. Blocking ACE reduces the cleavage, in turn reducing peripheral vascular resistance. This action reduces the myocardial oxygen consumption, thereby improving cardiac output and moderating left ventricular and vascular hypertrophy. Angiotensin II receptor blockers (ARBs) block the binding of angiotensin II (AII) to the AT II or (AII) receptor. ACE inhibitors and ARBs are thus used clinically (alone and together) to decrease arterial pressure, ventricular afterload, blood volume and hence ventricular preload, as well as inhibit and reverse cardiac and vascular hypertrophy.

ACE inhibitors are a first line therapy for most patients with CHF due to left ventricle systolic dysfunction. Many ACE inhibitors have been well studied in clinical trials and have been shown to reduce the rate of death and hospitalizations in individuals with heart failure. As symptoms progress, additional therapy consisting of a diuretic, a beta-blocker, a vasodilator, and possibly digoxin may be indicated.

The ARBs are a newer class of drugs commonly used to treat hypertension. By blocking angiotensin II, ARBs help relax and dilate the blood vessels, lowering blood pressure and decreasing the heart's workload, two important goals of treating heart failure.

Even with the use of ACE inhibitors and ARBs, quality of life for CHF sufferers is poor and worse than some other chronic diseases such as diabetes and chronic lung disease [J McMurray, H J Dargie. Diagnosis and management of heart failure. British Medical Journal 1994 308: 321-8.]. CHF mortality ranges from 50% over 5 years in mild heart failure [P A McKee, W P Castelli, P A McNamara, W B Kannel. The natural history of congestive heart failure: the Framingham study. New England Journal of Medicine 1971 285: 1441-6.] to 60% per year in severe cases [CONCENSUS Trial Study Group. Effects of enalapril on mortality in severe congestive heart failure. New England Journal of Medicine 1987 316: 1429-35.]; these figures are higher than breast and prostate cancer death rates. The CHF problem will increase (the so-called heart failure epidemic) because of the impact of treatment on other forms of heart disease (for example thrombolysis) and the ageing population [J G F Cleland. Heart failure: the epidemic of the millennium. Hospital Update 1994 January; 9-10.].

More recently, clinical trials have been initiated looking at the regenerative capacity of autologous hematopoietic stem cells when introduced into damaged tissue of a failing organ. See [Burt R, et al., Hematopoietic stem cell transplantation for cardiac and peripheral vascular disease 2003; Rafii S, Lyden D Therapeutic stem and progenitor cell transplantation for organ vascularization and regeneration]. Recent studies have suggested that marrow and blood hematopoietic stem cells may contribute to nonhematopoietic tissue repair in multiple organ systems. In animal models and more recently in limited human trials, unpurified marrow mononuclear cells and/or subsets of adult hematopoietic stem cells have been reported to contribute to neoangiogenesis. Recent preclinical and pioneering clinical studies have shown that introduction of bone marrow-derived endothelial and hematopoietic progenitors can restore tissue vascularization after ischemic events in limbs, retina and myocardium.

The present invention provides methods for improving the efficacy of introduction of hematopoietic stem cells in patients undergoing therapy with either ACE inhibitors or ARBs alone or in combination.

### SUMMARY OF THE INVENTION

An intermittent drug delivery protocol is disclosed whereby a drug or combination of drugs are administered over a given period of time. This is followed by a recovery period of time where the drug is not administered to the patient. Thereafter the drug or group of drugs are again administered to the patient. The alternating periods of drug administration followed by not administering the drug are continued for as long as the patient is being treated which may be for the entire life of the patient. The periods during which drug is administered can vary e.g., 1, 2, or 3 weeks, 1, 2, or 3 months or more and the periods during which drug is administered can vary e.g. 1, 2, or 3 weeks, 1, 2, or 3 months or more an may be the same as, longer than or shorter than the periods for which it is administered. The drug administered may be an angiotensin converting enzyme (ACE) inhibitor and/or an Angiotensin II receptor blocker (ARB). The periods of (1) drug administration and (2) recovery may be determined based on statistically results, or adjusted based on determined levels of HSC's in the patient.

A method of treatment is disclosed whereby a therapeutically effective amount of a drug which blocks activation of the renin-angiotensin system (RASBs), (including a combinations of such drugs) is administered to a patient wherein the drug is one which decreases the number and/or activity of hematopoietic stem cells (HSC) in the patient. The decrease in numbers or activity of the HSC may be determined by monitoring the patient's ACE⁺ HSC levels. To maximize efficacy of the introduction of HSCs to an ailing organ, the patients are changed to a dosage regime whereby 1) the levels and/or activity of ACE⁺ HSC cells are determined; 2) the patient ceases administration of the renin-angiotensin system blocker (RASB) to allow recovery of ACE⁺ HSC; 3) isolated ACE⁺ HSC are administered to the patient; and 4) the patient continues administration of the RASB. HSC levels following RASB may be monitored to identify an appropriate recovery period for a patient, and the administration of the ACE⁺ HSC cells may be timed to follow this recovery in a patient-specific manner. Alternatively, the administration of the ACE⁺ HSC cells may follow a prescribed period of time and dosing in each patient.

The HSC levels may be monitored prior to the patient receiving the initial dosage of the RASB to set a baseline level of ACE⁺ HSC, and the levels monitored throughout the treatment regime to help set the appropriate levels of drug to maintain activity but prevent associated anemia.

Following administration of the HSC, and sufficient time for the cells to promote recovery of damaged tissue in the organ, the drug can be readministered using the same dosage regime as before the cessation, or a revised regime based on the outcome of the therapeutic HSC intervention. Optionally, the patient can be monitored at specific points and when a determination is made that the patient's HSC levels are sufficiently improved the drug is again administered to the patient whereby the patient receives the benefit of ACE inhibitor without the adverse effect of the HSC levels being reduced to undesirably low levels, e.g. the levels and/or activity of ACE⁺ are maintained at a desirable level.

The RASB is preferably an ACE inhibitor, an ARB, or a combination thereof. The HSC levels may be monitored by detecting levels of angiotensin converting enzyme positive (ACE⁺) cells in the patient's peripheral blood. A secondary marker (e.g., CD34) may also be used to determine the relative populations of HSC present in the patient following administration of the RASB.

An aspect of the invention is a method of treatment whereby a RASB is administered for a given period of time, followed by a recovery period where no (or reduced amounts of) RASB are delivered, followed by again administering the RASB.

Another aspect of the invention is the treatment of organ failure such as CHF by the intermitant administration of a RASB in a treatment protocol which obtains beneficial effects of a RASB while reducing their adverse effects such as reduced HSC levels.

Yet another aspect of the invention is a method of treating a patient suffering from CHF while optimizing the efficacy of the intervention by providing an increased number of HSC with ACE⁺ activity.

An aspect of the invention is a method of treating a patient suffering from renal failure while optimizing the efficacy of the intervention by providing an increased number of HSC with ACE⁺ activity.

Yet another aspect of the invention is a method of treating a patient suffering from respiratory failure while optimizing the efficacy of the intervention by providing an increased number of HSC with ACE⁺ activity.

Another aspect of the invention is an intermittent treatment protocol for the administration of ACE inhibitors (alone or in combination) in conjunction with administration of HSC.

In another aspect of the invention is an intermittent treatment protocol for the administration of ARBs (alone or in combination) in conjunction with administration of HSC.

Another aspect of the invention is an intermittent treatment protocol for the administration of a RASB in conjunction with administration of HSC.

These and other aspects of the invention will become apparent to those skilled in the art upon reading this disclosure in combination with the figures attached hereto.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a schematic drawing showing the pathways by which ACE inhibitors work to block the cleavage of angiotensin I.

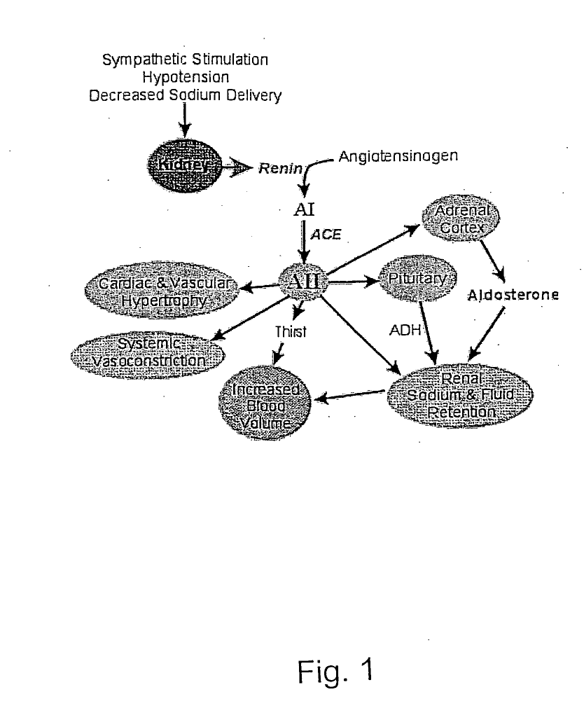

### DETAILED DESCRIPTION OF THE INVENTION

Before the present methods of treating and monitoring patients are described, it is to be understood that this invention is not limited to particular drugs or methods of monitoring described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a drug” includes a plurality of drugs, reference to “an ACE inhibitor” includes a plurality of such ACE inhibitors and reference to “the method of monitoring” includes reference to one or more methods of monitoring and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The terms “excipient material” “carrier” and the like are intended to mean any compound forming a part of a formulation which is intended to act merely as a filler, carrier or the like i.e. not intended to have biological activity itself.

The term renin-angiotensin system blocker, “RASB” and the like refer to any molecule which prevents the activation of the renin-angiotensin system and includes ACE inhibitors, and angiotensin II receptor blockers (ARBs) alone or in any combination.

The terms “treating”, and “treatment” and the like are used herein to generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a cell, group of cells, animal, mammal, and particularly a human, and includes: (a) preventing the disease from occurring in a subject which may be predisposed to the disease but has not yet been diagnosed as having it; (b) inhibiting the disease, i.e. arresting it's development; or (c) relieving the disease, i.e. causing regression of the disease and/or it's symptoms or conditions. The invention is directed towards treating patient's suffering from organ failure, including CHF, renal failure, and respiratory failure, over long periods of time. The present invention is involved in preventing, inhibiting, or relieving adverse effects attributed to organ failure and/or low levels of hematopoetic stem cells (HSC)—particularly over long periods of time. More specifically, treatment involves the intermittent administration of a drug which blocks activation of the renin-angiotensin system (RASBs) in a protocol which reduces effects on lowering the patient's HSC levels.

The terms “synergistic”, “synergistic effect” and the like are used interchangeably herein to describe improved treatment effects obtained by combining one or more different drugs with the method of the invention. Although a synergistic effect in some fields means an effect which is more than additive (e.g., one plus one equals three) in the field of treating CHF and related diseases an additive (one plus one equals two) or less than additive (one plus one equals a number greater than 1) effect may be synergistic. For example, if a patient has CHF and low levels of HSC, treatment to relieve the CHF and maintain higher levels of HSC above levels determined without the method of the invention is a synergistic result. In connection with the present invention co-administration of formulations of RASB drugs such as ACE inhibitors alone or in combination with ARBs with the method of the invention whereby HSC are monitored and treatment intermittently administered based on HSC levels makes it possible to obtain improved effects which are synergistic, i.e. greater than the effects obtained by the administration of a RASB continuously on two or more RASB drugs continuously.

### THE INVENTION IN GENERAL

Hematopoeitic stem cells are known to express ACE on their surface (see U.S. Pat. No. 6,610,497 issued Aug. 26, 2003, and PCT published application WO 98/23773 published Jun. 4, 1998) and a local renin-angiotensin system is involved in the microenvironment of the hematopoietic stem cell niche. The ability of a patient's hematopoietic system to aid in the regeneration of cardiac function is dependent in part on the ability of their hematopoietic stem cells to undergo cell division and differentiation. An ACE inhibitor blocks the ability of the hematopoietic stem cells to enter into S-phase, in effect blocking the ability of these cells to divide and produce other cell types necessary for the regeneration.

In connection with the present invention it has been recognized by the inventors that with CHF patients undergoing ACE-inhibitor therapy, the benefits of the therapy in terms of control of hypertension and relief of load on the heart is offset by loss in regenerative capability and reduced levels of HSCs. However, the adverse effects of ACE inhibitors are reversible. Thus, in accordance with the present invention the hematopoietic capacity can be monitored both with and without the drug, i.e. monitored over periods of time when one or more drugs are administered (treatment period) and periods of time when the drug is not administered (recovery periods). Identification of ACE⁺ cell levels in mobilized hematopoietic stem cells makes it possible to identify how long each period should be. The regenerative therapy may be merely temporarily stopping the administration of ACE inhibitor therapy and/or ARBs i.e. providing for a recovery period where no drug is administered. However, the recovery period may include injection of isolated hematopoietic stem cells into the circulatory system or directly into the patient's heart. Thus, identification of ACE levels, or levels of ACE activity, can be used as a monitoring methodology to determine the best time to treat a patient.

The identification of ACE⁺ cells may also be a surrogate marker for patients undergoing treatment with ARBs, as the blockage of the ARBs (particularly when combined with the administration of an ACE inhibitor) can lead to a decrease in the number of hematopoietic progenitor cells for use in regenerative therapy. Since ACE is a marker for such cells, identification of ACE⁺ cells can also be used to monitor the levels of hematopoiesis in patients undergoing ARB and/or ACE inhibitors therapy, and can be used to make treatment decisions such as those discussed.

Thus, identification of ACE⁺ cells is used in accordance with the present invention to aid directly in therapeutic decisions, such as 1) the decision to cease treatment with ACE inhibitors and/or ARB's for a period prior to regenerative intervention; 2) monitoring the level of ACE⁺ cells in such patients to identify recovery of ACE⁺ cell levels, and appropriate timing for introduction of HSC to the patient; and 3) identification of “responders”, i.e. people who will respond to therapeutic intervention versus patients without the appropriate regenerative capacity.

ACE inhibitors that may be used in conjunction with the invention include, but are not limited to, lisinopril (Zestril™), ramipril (Tritace™), trandolapril (Odrik™), captopril (Capoten™), fosinopril (Monopril™), enalapril (Renitec™), benazepril (Lotensin™), quinapril (Accupril™), and/or perindopril (Coversyl™). ARBs that may be used in conjunction with the invention include, but are not limited to, candesartan (Atacand™), telmisartan (Micardis™), irbesartan (Avapro™), losartan (Cozaar™), valsartan (Diovan™), olmesartan (Benicar™), and/or eprosartan (Teveten™).

Anaemia from a variety of causes is relatively common in older patients who are the majority of recipients of ACE-inhibitor therapy and the exact cause is not always clear. Thus, identification of ACE⁺ cells is also used in connection with the present invention to confirm or exclude such therapy as the cause of the anaemia.

The present invention is being described primarily for determination of regenerative capacity in treating patients with CHF. However, determining and monitoring can be used in the treatment of other chronic diseases such as chronic obstructive pulmonary disorder or end stage renal failure.

The intermittent treatment method of the invention makes it possible to obtain the benefit of drug treatment while reducing the adverse side effects of the drugs. The method may be carried out by the daily administration of either or both of an ACE inhibitor and an ARB during a treatment period. Thereafter, either one or both the ACE inhibitor and ARB drug are not administered during a recovery period. Both the treatment period and recovery period last a plurality of days, weeks or months. During the recovery period the patient may be treated with the administration of a formulation of HSCs. Thus, in accordance with an example, the method of the invention can be carried out as follows:

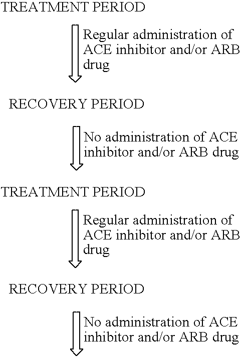

Both the treatment period and the recovery period can be for 1, 2, or 3 weeks, 1, 2, or 3 months and may be repeated many times over the life of the patient. Regular administration during this treatment period may be a dosage of the drug once daily, twice daily, once every other day, or as would be prescribed for regular use by a clinician skilled with dosage of the drug for treatment of the specific failing organ. The periods can be the same or different from each other in length. The method can be modified to include a step of monitoring levels of HSCs in the patient as follows:

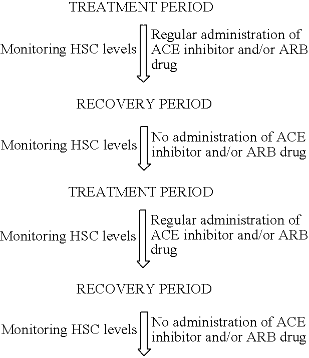

The monitoring step is carried out during the treatment period to detect when the patient's HSC level falls below a given desired point, e.g., the HSC levels have decreased a given percentage from pretreatment levels. When that level is reached the recovery period is started, and the regular dosage of the drug ceases for this period. Monitoring during the recovery period is carried out to determine the point when the patient's HSC level rises back to normal or to an acceptable point. When that point is reached the treatment period begins again. This intermittent treatment can be carried out a plurality of times and may be carried out for the patient's entire life if needed.

The method can be carried out on a large number of patients in clinical trials with monitoring. The monitoring data can be used statistically to determine the best length for both the treatment period and the recovery period. The periods may vary based on the age, condition, sex, weight and/or other parameters such the patient's HSC levels prior to any treatment.

This intermittent treatment methodology of the invention may be modified to be an alternating two types of treatment. This is carried out by adding the administration of a formulation of HSCs during the recovery period as follows:

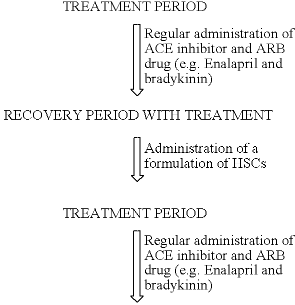

The recovery period may be shortened considerably by administering a HSC formulation as compared to a recovery period when no HSC formulation is administered.

### TREATING ORGAN FAILURE WITH DRUGS

The renin-angiotensin system plays an important role in regulating arterial the function of numerous organ systems, including cardiac and vascular function and kidney function. Renin is an enzyme that acts upon a circulating substrate, angotensiongen, which undergoes proteolytic cleavage to form the angiotension I decapeptide. Angiotensin converting enzyme (ACE) converts the octapeptide angiotensin I to angiotensin II (AII) by cleavage of dipeptide.

For example, M. R. Celio, et al. [Proc. Natl. Acad. Sci. USA, 78, 3897 (1981) and Histochemistry, 72, 1 (1981)] have identified specific glomerular sites of A II production from angiotensin I (A I) and have demonstrated its subsequent blockage by the ACE inhibitor compound, enalapril.

Both ACE inhibitors and ARBs have been shown to suppress hematopoiesis in different patient populations particularly when administered in combination.

ACE is believed to enhance the recruitment of early hematopoietic progenitor cells into S-phase in the bone marrow. ACE inhibitors have been shown to reduce the circulating hematopoietic progenitors in healthy subjects. Certain ACE inhibitors have been shown to impact directly on the function of the hematopoietic system, in a manner that can be reversed upon ceasing administration of the drug. For example, the ACE inhibitor Enalapril was shown to cause reversible anemia in renal transplant patients. Vlahakos et al., Enalapril-associated amenia in renal transplant recipients treated for hypertension, Am J Kidney Dis.; 17:199-205 (1999). The present invention provides a solution to the problem that ACE inhibitors, as well as being vasodilators, inhibit production of blood cells, and acts as direct inhibitors of hematopoiesis.

ARBs have also been shown to reversibly inhibit hematopoiesis in patients taking this class of drugs. The drug Losartan has been shown to reversibly decrease haemoglobin and hematocrit levels in patients with chronic obstructive pulmonary disease. Vlahakos, D V and Kosmas, E N. Losartan Reduces Hematocrit in Patients with Chronic Obstructive Pulmonary Disease and Secondary Erythrocytosis, Annals of Internal Medicine; 134:426-7 (2001). It is not clear whether the effect is due to action solely within the same pathways as ACE, or whether there is redundancy in the pathways involved in this inhibitory activity.

While not committing to any particular mechanism of action it is proposed that a local renin-angiotensin system (RAS) is active in the bone marrow, and that this plays a role in regulating hematopoiesis. The RAS is thought to be an important determinate for erythropoiesis. RAS inactivation may confer susceptibility to hematocrit lowering effects of ACE inhibitors and ARBs. It is not clear whether it will also impact upon the vascularization ability of hematopoietic stem cells.

### IDENTIFYING HEMATOPOIETIC PROGENITOR CELLS

The marker ACE along with the marker CD34 have the ability to identify and isolate an early hematopoietic progenitor cell. ACE binding agents may be used to isolate hematopoietic progenitors. As ACE can be used to preferentially isolate the hematopoietic progenitor populations with increased regenerative capacity, it can be used in connection with the present invention for identifying hematopoietic progenitors for many purposes, including the traditional use in bone marrow transplantation. This may increase the efficiency of transplantation by identifying a preferred transplantation cell population.

It is possible to detect HSC via methods disclosed in published PCT application WO 03/038071 published May 8, 2003 as well as the various publications cited therein. In this method a sample comprising HSC or progeny thereof is obtained. In the sample the presence of at least one carbohydrate sequence comprising a sequence of at least one disaccharide repeat of glucosome acid and N-acetylglucosamine or an equivalent thereof and identifying a HSC with that sequence. The HSC can be isolated as described in the WO 03/038071 application and publications cited therein all of which are incorporated herein.

As used herein, the terms “antibody” or “antibodies” include the entire antibody and antibody fragments containing functional portions thereof. The term “antibody” includes any monospecific or bispecific compound comprised of a sufficient portion of the light chain variable region and/or the heavy chain variable region to effect binding to the epitope to which the whole antibody has binding specificity. The fragments can include the variable region of at least one heavy or light chain immunoglobulin polypeptide, and include, but are not limited to, Fab fragments, F(ab′)₂ fragments, and Fv fragments.

The recombinant antibody can be produced by any recombinant means known in the art. Such recombinant antibodies include, but are not limited to, fragments produced in bacteria and non-human antibodies in which the majority of the constant regions have been replaced by human antibody constant regions. In addition, such “humanized” antibodies can be obtained by host vertebrates genetically engineered to express the recombinant antibody.

In addition, the monospecific domains can be attached by any method known in the art to another suitable molecule compound. The attachment can be, for instance, chemical or by genetic engineering.

The antibodies can be conjugated to other suitable molecules and compounds including, but not limited to, enzymes, magnetic beads, colloidal magnetic beads, haptens, fluorochromes, metal compounds, radioactive compounds, chromatography resins, solid supports or drugs.

Examples of the enzymes that can be conjugated to the antibodies include, but are not limited to, alkaline phosphatase, peroxidase, urease and β-galactosidase.

Examples of the fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies see Haugland, R. P. Molecular Probes: Handbook of Fluorescent Probes and Research Chemicals (1992-1994).

Examples of the metal compounds that can be conjugated to the antibodies include, but are not limited to, ferritin, colloidal gold, and particularly, colloidal superparamagnetic beads.

Examples of the haptens that can be conjugated to the antibodies include, but are not limited to, biotin, digoxigenin, oxazalone, and nitrophenol.

Examples of the radioactive compounds that can be conjugated or incorporated into the antibodies are known to the art, and include but are not limited to technetium 99m, ¹²⁵I and amino acids comprising any radionuclides, including, but not limited to ¹⁴C, ³H and ³⁵S.

The antibodies to the cell surface HSC marker may be obtained by methods known in the art for production of antibodies or functional portions thereof. Such methods include, but are not limited to, separating B cells with cell-surface antibodies of the desired specificity, cloning the DNA expressing the variable regions of the light and heavy chains and expressing the recombinant genes in a suitable host cell. Standard monoclonal antibody generation techniques can be used wherein the antibodies are obtained from immortalized antibody-producing hybridoma cells. These hybridomas can be produced by immunizing animals with HSCs or progeny thereof, and fusing B lymphocytes from the immunized animals, preferably isolated from the immunized host spleen, with compatible immortalized cells, preferably a B cell myeloma.

However, the present invention may be used to distinguish between various phenotypes of the HSC population including, but not limited to, the ACE⁺, CD34⁺, CD38⁻, CD90⁺ (thy1) and Lin⁻ cells. Preferably the cells identified are selected from the group including, but not limited to, ACE⁺, CD34⁺, CD38⁻, CD90⁺ (thy 1), or Lin⁻ all of which are protein markers well known to those skilled in the art reading this disclosure.

Various techniques can be employed to separate or enrich the cells by initially removing cells of dedicated lineage. Monoclonal antibodies and binding proteins are particularly useful for identifying cell lineages and/or stages of differentiation. The antibodies can be attached to a solid support to allow for crude separation. The separation techniques employed should maximize the retention of viability of the fraction to be collected. Various techniques of different efficacy can be employed to obtain “relatively crude” separations. The particular technique employed will depend upon efficiency of separation, associated cytotoxicity, ease and speed of performance, and necessity for sophisticated equipment and/or technical skill.

Procedures for separation or enrichment can include, but are not limited to, magnetic separation, using antibody-coated magnetic beads, affinity chromatography, cytotoxic agents joined to a monoclonal antibody or used in conjunction with a monoclonal antibody, including, but not limited to, complement and cytotoxins, and “panning” with antibody attached to a solid matrix, e.g., plate, elutriation or any other convenient technique.

The use of separation or enrichment techniques include, but are not limited to, those based on differences in physical (density gradient centrifugation and counter-flow centrifugal elutriation), cell surface (lectin and antibody affinity), and vital staining properties (mitochondria-binding dye rho123 and DNA-binding dye, Hoescht 33342).

Techniques providing accurate separation include, but are not limited to, FACS, which can have varying degrees of sophistication, e.g., a plurality of color channels, low angle and obtuse light scattering detecting channels, impedence channels, etc. Any method which can isolate and distinguish these cells according to levels of expression may be used.

Any separation methods employing antibodies to isolate cells may be utilised and are familiar to the skilled in the art reading this disclosure. The description above for identification of HSCs or progeny thereof is applicable here.

To further enrich for any cell population, specific markers for those cell populations may be used. For instance, specific markers for specific cell lineages such as lymphoid, myeloid or erythroid lineages may be used to enrich for or against these cells. These markers may be used to enrich for HSCs or progeny thereof by removing or selecting out mesenchymal or keratinocyte stem cells which isolated cells can be formulated and administered in accordance with the present invention.

The methods described above can include further enrichment steps for cells by positive selection for other stem cell specific markers. Suitable positive stem cell markers include, but are not limited to, ACE⁺, CD34⁺, Thy-1⁺, and c-kit⁺. By appropriate selection with particular factors and the development of bioassays which allow for self-regeneration of HSCs or progeny thereof and screening of the HSCS or progeny thereof as to their markers, a composition enriched for viable HSCs or progeny thereof can be produced for a variety of purposes.

### IDENTIFICATION OF ORGAN FAILURE

The intermittent dosage regime of the invention can be used for enhancing the activity of multiple failing organs, with the actual regime, dosage, and route of administration of both the drugs and the HSC dependent on the nature of the organ, the extent of the damage, and the availability of ACE⁺ HPC available for use in the patient. It is well within the skill of one in the art, upon reading the present disclosure, to optimize the dosage and route of administration for the particular organ to be treated. In addition, the condition and physical attributes of the patient (e.g., weight, age and the like) will need to be taken into consideration when determining the optimal dosages and/or routes of administration.

Cardiac Failure

Congestive heart failure (CHF) is generally the end result of cardiac disease, hypertension, or an eventual consequence of the collective effects of more acute forms of heart damage. The majority of products available for treatment of CHF do not directly address the underlying failure of the cardiac tissue but seek to relieve the burden on the heart and ameliorate the fluid build up associated with heart failure.

Patients with left ventricular dysfunction present to the physician either with symptoms of decreased exercise tolerance, a syndrome of fluid retention; or incidentally discovered left ventricular dysfunction. The development of heart failure is frequently a slow process, which may take years to be recognized as symptomatic disease.

The first steps in evaluating the structural abnormality and cause responsible for the development of heart failure are a detailed history and physical examination. The presence or absence of circulatory congestion, valvular insufficiency and in some cases congenital abnormalities can be evaluated at the time of initial contact with the patient, which leads to further investigations.

Although the history and physical examination may provide important clues about the underlying cardiac abnormality, identification of the structural abnormality leading to heart failure generally requires imaging of the cardiac structures.

The single most useful diagnostic test evaluating patients with suspected heart failure is the 2-dimensional echocardiogram, coupled with Doppler flow studies. With the help of echocardiography the degree of left ventricular systolic dysfunction can be determined which has important prognostic implications. Doppler echocardiography assists in the diagnosis of diastolic dysfunction.

Both chest radiography and 12-lead electrocardiograms provide baseline information in most patients, but as both are relatively insensitive and nonspecific, they usually do not form the primary bases for determining the specific cardiac abnormality responsible for the development of heart failure.

Recently the measurement of circulating levels of brain natriuretic peptide (NT-proBNP by Roche diagnostics) has become available as a means of identifying patients with elevated left ventricular filling pressures who are likely to have signs and symptoms of heart failure. Brain naturetic peptide (BNP) belongs to a group of naturetic peptides that are involved in the regulation of diuresis and which antagonise the vasoconstrictor effects of the reninangiotensin-aldosterone system (RAAS). Studies have shown that already in early phases of left-ventricular dysfunction (asymptomatic left ventricular systolic dysfunction and diastolic dysfunction) BNP proved to be a good marker of both development of heart failure and prognosis.

Other criteria for diagnosis of cardiac failure include bradycardia (heart rate <50 bpm); Hypotension (mean arterial pressure of less than 50 mmHg); ventricular tachycardia or fibrillation; and metabolic acidosis (pH <7.2).

Respiratory Failure

Respiratory failure may be classified as hypoxemic or hypercapnic and may be either acute or chronic.

Hypoxemic respiratory failure (type I) is characterized by a PaO₂ of less than 60 mm Hg with a normal or low PaCO₂. This is the most common form of respiratory failure, and it can be associated with virtually all acute diseases of the lung, which generally involve fluid filling or collapse of alveolar units. Examples of type I respiratory failure include cardiogenic or noncardiogenic pulmonary edema, pneumonia, and pulmonary hemorrhage.

Hypercapnic respiratory failure (type II) is characterized by a PaCO₂ of more than 50 mm Hg. Hypoxemia is common in patients with hypercapnic respiratory failure who are breathing room air. The pH depends on the level of bicarbonate, which, in turn, is dependent on the duration of hypercapnia. Common etiologies include drug overdose, neuromuscular disease, chest wall abnormalities, and severe airway disorders (eg, asthma, chronic obstructive pulmonary disease [COPD]).

The criteria for diagnosis of respiratory failure include: a respiratory rate of either less than five or greater than 40 breaths per minute; hypercapnia (PaCO₂ of greater than 6.7 kPa); and hypoxaemia.

Renal Failure

Acute renal failure is defined as an abrupt cessation or substantial reduction of renal function and, in as many as 90-95% of cases, may be secondary to trauma, surgery or another acute medical condition. Acute renal failure may be due to pre-renal causes (e.g., decreased cardiac output, hypovolemia, altered vascular resistance) or to post-renal causes (e.g., obstructions or constrictions of the ureters, bladder or urethra) which do not directly involve the kidneys and which, if treated quickly, will not entail significant loss of nephrons or other damage to the kidneys. Alternatively, acute renal failure may be due to intrinsic renal causes which involve a more direct insult or injury to the kidneys, and which may entail permanent damage to the nephrons or other kidney structures. Intrinsic causes of acute renal failure include but are not limited to infectious diseases (e.g., various bacterial, viral or parasitic infections), inflammatory diseases (e.g., glomerulonephritis, systemic lupus erythematosus), ischemia (e.g., renal artery occlusion), toxic syndromes (e.g., heavy metal poisoning, side-effects of antimicrobial treatments or chemotherapy), and direct traumas.

The diagnosis and treatment of acute renal failure is as varied as its causes. In human patients, oliguria (urine output <400 ml/day) or anuria (urine output <50 ml/day) may be present in 50-70% of cases, BUN levels may climb 10-20 mg/dl/day or faster, plasma creatinine levels may climb 0.5-1.0 mg/dl/day, and metabolic acidosis is almost always present. If not treated, the electrolyte and fluid imbalances (e.g., hyperkalemia, acidosis edema) associated with acute renal failure may lead to life-threatening arrhythmia, congestive heart failure, or multiple organ system failures. Present therapies are typically directed at the underlying causes of the acute renal failure (e.g., pre-renal, post-renal, or infectious causes) and management of the complications. Due to the severity of acute renal failure, episodes rarely last longer than several weeks without mortality and are treated on an in-patient basis.

Chronic renal failure may be defined as a progressive, permanent and significant reduction of the glomerular filtration rate (GFR) due to a significant and continuing loss of nephrons. Chronic renal failure typically begins from a point at which a chronic renal insufficiency (i.e., a permanent decrease in renal function of at least 50-60%) has resulted from some insult to the renal tissues which has caused a significant loss of nephron units. The initial insult may or may not have been associated with an episode of acute renal failure. Irrespective of the nature of the initial insult, chronic renal failure manifests a “final common path” of signs and symptoms as nephrons are progressively lost and GFR progressively declines. This progressive deterioration in renal function is slow, typically spanning many years or decades in human patients, but seemingly inevitable.

The functions of a kidney can be evaluated by one of several indexes, i.e., an excreting function, which is one of the most important functions. The index of the excreting function is, usually, an endogenous creatinine clearance (Ccr) that corresponds nearly to an amount of a glomerular filtration. Ccr indicates a renal excreting function for a creatinine that is a metabolite of a muscle, and can be regarded as a representative or standard value of the excreting function of a kidney. A normal value of Ccr is 70 to 130 mL/min.

A urine volume is also used as one of the parameters reflecting renal functions, because it generally is decreased with a decrease of renal functions (except for cases wherein a urine volume is temporarily increased when an abnormality in a filtrating function of a kidney is caused), and in particular, is remarkably decreased in an end-stage renal failure. A normal value of a urine volume is 1000 to 1500 mL/day.

The Ccr decreases with a progress of a chronic renal dysfunction, such as chronic glomerulonephritis, diabetic nephropathy, or nephrosclerosis. In general, a state having a Ccr value of 30 mL/min or less is called a chronic renal failure. After a pathologic state reaches such a chronic renal failure, a renal function, i.e., a residual renal function, cannot be recovered, and ultimately the pathologic state reaches a state of uremia. A serious state having a decreased Ccr value of 10 mL/min or less is called uremia. After a pathologic state reaches uremia, a urine volume falls generally to 1000 mL/day or less. As above, in a pathologic state of a chronic renal failure, the Ccr and the urine volume gradually decrease with a deterioration of a residual renal function, and when the pathologic state is worsened, the Ccr and the urine volume cannot be recovered.

In patients suffering from chronic renal failure, the formulations of the invention are preferably injected into the patient at or near the area of damage, e.g., into the bloodstream near the damaged kidney or directly into the area of damage.

Hepatic Failure

Acute liver failure (ALF) is a broad term that refers to both fulminant hepatic failure (FHF) and subfulminant hepatic failure (or late-onset hepatic failure). The latter term is reserved for patients with liver disease for up to 26 weeks prior to the development of hepatic encephalopathy. Some patients with previously unrecognized chronic liver disease decompensate and present with liver failure; although this technically is not FHF, discerning this at the time of presentation may not be possible (eg, Wilson disease).

FHF is a term used to describe the development of coagulopathy and encephalopathy as a result of acute hepatic decompensation within 8 weeks from the onset of illness. FHF may result from a variety of hepatic disease processes. Viral hepatitis and hepatotoxic drugs are the most common causes of FHF. Previously, a lower proportion of cases of FHF were attributed to acetaminophen, but recently, acetaminophen toxicity has become a more common cause. Acute acetaminophen hepatotoxicity leads to FHF in a variable proportion of patients. Treatment of the underlying process is essential, but the common factor underlying the severity of illness is loss of hepatic function.

Chronic hepatic failure occurs where there is deterioration in liver function superimposed on chronic liver disease. The acute deterioration may be based on a number of pathological processes, including the underlying disease process itself or a different process which undermines the functional reserve of the liver such as infection, hemorrhage or electrolyte imbalance—especially hypokalaemia.

The actual deterioration in the patient's state may occur very rapidly—the patient may progress through confusion and stupor into coma in a matter of hours. The criteria for diagnosis of hepatic failure include detection of coagulation defects and rising hepatic enzymes, including hypoalbuminaemia.

### INJECTING ISOLATED ACE⁺ CELLS

In accordance with the methodology such as that described above it is possible to isolate ACE⁺ cells and concentrate those cells into a formulation. The concentrated cells can be formulated into an injectable formulation and then injected into a patient. For example, the cells can be injected into damaged myocardium. Preferably, the cells are extracted from a patient, concentrated, formulated, and injected back into the same patient so that the cells are autologous. The ACE⁺ cells administered to the patient may also be allogeneic or derived from an alternate cell source, e.g., cells differentiated from embryonic stem cells.

In accordance with one aspect of the invention the cells are formulated in a matrix. By dispersing the ACE⁺ cells within the matrix material and then injecting the material into the patient such as the patient's heart, muscle, or other tissue material improved treatment effects can be obtained. The matrix may be a biological material which may be a non-cellular material which aids in tissue regeneration. Non-cellular matrix compositions are disclosed in a number of patents such as U.S. Pat. Nos. 4,902,508; 4,956,178; 5,281,422; 5,275,826; 5,554,389 6,696,270; and 6,653,291 as well as numerous patents and publications cited in these patents. An acellular tissue matrix that has been used successfully in a number of applications is the matrix prepared by the methods disclosed in U.S. Publ. Nos. 2003/0035843 and 2003/0143207 to S. A. Livesey, et al., and are incorporated by reference herein in their entirety. The methods involve processing biological tissue with a stabilizing solution to reduce damage to the tissue, decellularizing the tissue, treating the decellularized tissue with a cryoprotectant solution, then freezing and drying the tissue. The processed acellular tissue may then be stored and, ultimately, rehydrated for use. The conditions employed in these methods minimize damage to the functionality of the tissue.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of treatment, comprising the steps of:
(a) administering to a patient a therapeutically effective amount of a pharmaceutically active drug, wherein such drug decreases the hematopoietic stem cells (HSC) in the patient;
(b) discontinuing the administering as in step (a) to allow recovery of the level of HSC; and
(c) administering the drug to a patient as per step (a) following recovery of HSC levels in the patient.

2. The method of claim 1, further comprising:
(d) monitoring the level of HSC following step (b) to determine recovery of HSC levels.

3. The method of claim 1, further comprising:
isolating HSC from the patient following recovery of the HSC; and
reintroducing the isolated HSC to the patient.

4. The method of claim 3, wherein the isolated HSC are delivered to an organ of the patient chosen from heart, kidney, liver and lung.

5. The method of claim 1, further comprising:
repeating the steps (a), (b) and (c) a plurality of times;
wherein the drug is a drug which blocks activation of the patient's renin-angiotensin systems (RASB).

6. The method of claim 1, wherein the drug is an Angiotensin Converting Enzyme (ACE) inhibitor.

7. The method of claim 1, wherein the drug is an Angiotensin II receptor antagonist.

8. The method of claim 3, wherein the drug is a combination of an Angiotensin Converting Enzyme (ACE) inhibitor and an Angiotensin II receptor antagonist.

9. The method of claim 1, wherein the pharmaceutically active drug is an Angiotensin Converting Enzyme (ACE) inhibitor.

10. The method of claim 1, wherein the pharmaceutically active drug is Angiotensin II receptor antagonist.

11. The method of claim 2, wherein the monitoring step (d) is carried out by detecting levels of Angiotensin Converting Enzyme positive (ACE⁺) cells in the patient's peripheral blood.

12. The method of claim 2, wherein the monitoring step (d) is carried out by detecting levels of a cell surface protein in the patient's peripheral blood wherein the protein is chosen from CD34⁺, CD38; CD90⁺ (thy1), and Lin⁻.

13. The method of claim 1, further comprising:
repeating the steps (a), (b) and (c) a plurality of times;
wherein the pharmaceutically active drug is a combination of an Angiotensin Converting Enzyme (ACE) inhibitor and an Angiotensin II receptor antagonist; and
further wherein the administering (a) is carried out by administering drug on a daily basis or more frequently for three weeks or more and the discontinuing (b) is carried out for three weeks or more.

14. A method of treatment, comprising the steps of:
(a) administering to a patient a therapeutically effective amount of a pharmaceutically active drug which blocks activation of the patient's renin-angiotensin systems (RASB);
(b) determining a level of hematopoietic cells in the patient;
(c) discontinuing the administering as in step (a) when the level of hematopoietic cells as determined in step (b) drops below a given level;
(d) continuing the administering as in step (a) when the level of hematopoietic cells as determined in step (b) rises above a given level.

15. The method of claim 14, wherein the pharmaceutically active drug decreases the concentration of hematopoietic cells in the patient.

16. The method of claim 15, wherein the pharmaceutically active drug is an Angiotensin Converting Enzyme (ACE) inhibitor.

17. The method of claim 15, wherein the pharmaceutically active drug is Angiotensin II receptor antagonist.

18. The method of claim 14, wherein the determining step (b) is carried out by detecting Angiotensin-converting Enzyme of hematopoietic cells.

19. The method of claim 15, wherein the pharmaceutically active drug is a combination of an Angiotensin Converting Enzyme (ACE) inhibitor and an Angiotensin II receptor antagonist.

20. The method of claim 14, further comprising:
isolating HSC from a patient histocompatible with the patient; and
reintroducing the isolated HSC to the patient.

21. The method of claim 20, wherein the isolated HSC are delivered to an organ of the patient chosen from heart, kidney, liver and lung.

22. The method of claim 20, wherein the isolated HSC are administered to the same patient from which they are isolated.

23. A method of treating a patient, comprising the steps of:
monitoring hematopoietic stem cells (HSC) in a patient by determination of the patient's Angiotensin Converting Enzyme (ACE) level to obtain a monitored level; and
administering an ACE inhibitor to the patient based on the monitored level.

24. The method of claim 23, further comprising:
administering an Angiotensin II receptor antagonist to the patient based on the monitored level.

25. The method of claim 2, wherein the monitoring the level of HSC comprises the steps of:
obtaining a cell sample including HSC or progeny thereof;
combining the sample with a labeled antibody which binds a protein on a HSC surface or a fragment thereof;
detecting the presence of the protein; and
identifying the HSC or progeny thereof having the protein or a fragment thereof by detecting the presence of the label on the antibody bound to the protein.

26. The method of claim 25, wherein the protein is chosen from CD34⁺, CD38⁻, CD90⁺ , (thy1), and Lin⁻.

27. The method of claim 25, wherein the protein is Angiotensin Converting Enzyme (ACE).

28. The method of claim 3, wherein the isolating of HSC comprises the steps of:
obtaining a cell population comprising HSC or progeny thereof;
detecting the presence of a protein or a fragment thereof on a cell of the population; and
selecting for cells which are identified by the presence of the protein or a fragment thereof on the cell.

29. The method of claim 3, wherein the isolating of HSC comprises the steps of:
obtaining cell populations comprising HSCs or progeny thereof;
combining the cell population with a labeled antibody which binds a protein or a fragment thereof expressed on a surface of an HSC; and
selecting for cells which are identified by the presence of the label on the antibody binding the protein or a fragment thereof on the HSC.

30. The method of claim 29, wherein the protein is chosen from CD34⁺, CD38⁻, CD90⁺ , (thy1), and Lin⁻.

31. The method of claim 29, wherein the protein is Angiotensin Converting Enzyme (ACE).

32. A method of treatment for organ failure, comprising the steps of:
isolating angiotensin converting enzyme positive (ACE⁺) cells;
formulating the ACE⁺ cells into an injectable formulation; and
injecting a therapeutically effective amount of the formulation into a patient.

33. The method of claim 32, wherein the ACE⁺ cells are isolated from the blood of a patient suffering from Coronary Heart Failure (CHF).

34. The method of claim 33, wherein the patient suffering from CHF is the same patient injected with the formulation.

35. The method of claim 32, further comprising:
combining the ACE⁺ cells with an acellular matrix.

36. A method of treatment, comprising the steps of:
(a) administering to a patient a combination of an angiotensin converting enzyme (ACE) inhibitor and an angiotensin II receptor antagonist during a treatment period;
(b) discontinuing the administration of the combination to the patient; and
(c) readministering the combination to the patient.

37. The method of claim 36, wherein the treatment period is three weeks or more and the recovering period is three weeks or more.

38. The method of claim 36, wherein the combination is administered once a day or more frequently during the treatment period.

39. The method of claim 36, wherein (a), (b) and (c) are repeated a plurality of times.

40. The method of claim 39, wherein the length of the treatment period (a) and recovery period (b) are determined by monitoring the patient's HSC level.
